(19) United States
(12) Patent Application Publication
(10) Pub. No.: US 2007/0160695 A1
(43) Pub. Date: Jul. 12, 2007

(54) Pharmaceutical preparations for treating hypertension and dyslipidemia with Allium ursinum and Allium sativum

(76) Inventor: Dallas L. Clouatre, Santa Monica, CA (US)

Correspondence Address: DALLAS L CLOUATRE, 1223 WILSHIRE BLVD #761, SANTA MONICA, CA, 90403-5400, US

(21) Appl. No.: 11/328,540

(22) Filed: Jan. 9, 2006

(51) Int. Cl. A61K 36/8962

(52) U.S. Cl. 424/754

(57) ABSTRACT

Wild Garlic (Bear's or Forest Garlic, Allium ursinum) can be combined with cultivated high-allicin garlic (Allium sativum) and extracts of the same to take advantage of the previously unknown synergism of preparations made from these two garlic species. There is a substantial cost savings over the use of pure, Wild Garlic and an elimination of the side effects found with the chronic ingestion of high levels of cultivated allicin-rich garlic (Allium sativum). The resulting combination has demonstrated benefits with regard to blood lipids regulation in individuals in need thereof that are two or more times that expected from the use of either garlic species taken singularly. Blood pressure regulating benefits are maintained or improved, the odor-causing potential of high-allicin garlic is reduced, and significant improvements are found in the areas of total, LDL, and VLDL cholesterol and triglycerides, benefits far beyond the merely additive. HDL cholesterol levels also are improved. It is to be expected that these benefits, in addition, can be extended through the concurrent intake of folic acid, red yeast rice extracts and other compounds proposed for improving cardiovascular health. Still further, a variety of defined delivery approaches, including, but not limited to controlled releases, especially those including mucoadhesive, can be utilized to improve the effects taught herein.

### BACKGROUND OF THE INVENTION

1. Field of the Invention

The invention is directed toward a method of treatment of dyslipidemia and hypertension using Allium ursinum in a synergistic combination with Allium sativum. Appropriate delivery techniques, including the use of mucoadhesives (mucosal adhesives) are described.

2. Description of Prior Art

Mild-moderate hypertension is recognized as an important risk factor in heart diseases and stroke. Hypertension is poorly treated with normal pharmaceutical preparations because of low compliance and side effects. Animal studies in hypertensive rats have shown that garlic decreases systolic pressure by 20-30 mmHg and diastolic pressure by 10-20 mmHg in the hypertensive animals. Most importantly, when there was a significant drop in systolic and diastolic pressure, there was a significant decrease in heart rate in these animals in conjunction with this drop in their blood pressure. (Cytobios 1982;34: 145-162.) This is the opposite of what happens in humans with hypertension who are treated with many of today's antihypertensive medications. Animal studies also have shown a decrease in hydroxyproline concentrations in the rat myocardium after garlic treatment. This suggests a reduction in myocardial fibrosis.

Importantly, human trials have confirmed these antihypertensive findings. In one trial, subjects with initial diastolic pressures of 95-104 mmHg experienced reductions to 89 mmHg within 12 weeks using a high-allicin garlic preparation. (The British Journal of Clinical Practice 1990;Suppl. 69:3-6.) A recent meta-analysis of blood pressure improvements achieved with the same preparation as reported in 8 published studies concluded this garlic preparation might be of use in cases of mild clinical hypertension. The usual dose was 300 mg. two to three times per day or 600 to 900 mg. per day. (Journal of Hypertension 1994; 12 :463-468; Hämostaseologie 1993;13: 43-52.) Therefore, it has been shown that allicin-containing garlic preparations are clinically effective in lowering elevated blood pressure.

Similarly, the lipid lowering effects of garlic and onions has been confirmed many times since the now-famous 1979 population study of three sections of vegetarians in the Jain community in India. In that study, those who ate 50 grams of garlic and 600 grams of onion per week, showed total cholesterol levels of 159 mg./dL and triglyceride levels of 52 mg./dL. In contrast, consumption of 10 grams of garlic and 200 grams of onions led to total cholesterol levels of 172 mg/dL and triglyceride levels of 75 mg/dL. Those eating no garlic and onion showed total cholesterol levels of 208 mg./dL and triglyceride levels of 109 mg./dL. (Indian J. Medical Research 1979;69 :776-780; J. Ass. Phys. Ind. 1979;27:707-712.) Subsequent tests have shown a variety of compounds are at work. One mechanism of action is the reduction of cholesterol synthesis. This leads to re-uptake of total cholesterol by the liver and results in an increase in HDL cholesterol at the same time reducing LDL levels. Another mechanism of action is related specifically to the prevention of oxidation of LDL cholesterol. (Lipids 1993;28(5):475-477; Circulation Research 1990;66:311-320; Nutrition Research 1983;3:119-128.)

Despite the obvious health benefits of garlic supplementation, high-allicin garlic powders and extracts, and garlic powders and extracts that retain the potential to produce large amounts of allicin when rehydrated after ingestion suffer from numerous disadvantages.

First, they produce a sulfuracious breath odor as the metabolites of allicin are eliminated through the lungs. With dosages in the range of 900 mg. per day of the best quality powdered garlic products, as least 25% of the users will find the smell completely unacceptable despite using the extract under a doctor's orders. Even minimal dosages of the active oil-soluble principles of regular garlic, (as delivered by only 600 mg of extract per day) cause an accumulation of these elements within the body until a point is reached at which garlic odor appears as these principles are eliminated through the breath. Enteric coatings that include chlorophyll and other means have been tried to render ingested allicin odorless, but have met with little success. Nevertheless, allicin should be protected from the action of stomach acid and enzymes if it is to be absorbed at a high rate from low dosage forms. (Dallas Clouatre. European Wild Garlic: The Better Garlic. PAX Publishing, 1995.)

Second, ingested allicin and ingested extracts which produce allicin in the small intestine are damaging to the gastrointestinal tract if taken in any large quantity, which for a non-negligible percentage of patients is as low as 700- 900 mg per day. Even under a doctor's prescription, another 5% of users of A. sativum products will be unable to tolerate the GI tract distress which can accompany the ingestion of regular garlic. (Hämostaseologie 1993;13:43-52)

Third, allicin and its immediate transformation products tend to rupture red blood cells in the system. Any large amount of allicin entering the system can have minor toxic results.

Finally, to provide full protection against lipid synthesis, allicin-containing and allicin-potential-containing garlic powders and extracts must be taken several times per day. This is in sharp contrast with the blood pressure-lowering compounds found in garlic, most of which are in the water-soluble fraction, rather than the oil-soluble fraction. These hypotensive compounds are largely lost when products are concentrated for allicin or allicin-potential. (James M. Dunn and Vincent B. Ciofalo. Rocky Mountain Medical Journal September 1982.)

Several needs thus present themselves with regard to cultivated garlic (Allium sativum) products as treatment modalities for blood dyslipidemia and hypertension. There is need of a means of greatly increasing the lipid-lowering effect of cultivated allicin-containing garlic extracts such that much smaller amounts of allicin are required to achieve the same or greater effects. A quite surprising result of the combination of Wild or Bear's Garlic (Allium ursinum) with ordinary cultivated garlic (Allium sativum) is precisely this effect upon blood lipids. This is a surprising finding because, in part, according to published in vitro data (Sendl A., et al. Atherosclerosis 1992;94:79-95), Allium ursinum is nearly identical in efficacy to generic garlic in inhibiting cholesterol synthesis. Sendl, et al., p. 83, also refers to numerous clinical trials (all utilizing Allium sativum) showing a lowering of total cholesterol of up to 25%. In an animal trial, both types of garlic were found to similarly influence total cholesterol levels, with Wild Garlic proving to be slightly superior. (Preuss H G , Clouatre D, et al. Int Urol Nephrol. 2001;32(4):525-30.) Wild Garlic has been shown to be superior to high-allicin garlic on a weight/weight basis for lowering blood pressure. (Mohamadi A, Jarrell S T, Shi S J, et al. Heart Disease. 2000 January-February; 2(1):3-9.) There is a large overlap in the compounds found in the two species, and therefore it has been expected that they would be similarly efficacious on a weight/weight basis and that mixing the two species would yield, at most, a small additive benefits. In fact, the position of Sendl, et al. and other authorities knowledgeable and skilled in the art with extensive work with both species of garlic appears to be that the two species merely can be substituted for one another. It never has been suggest previously that the combination of the two might be particularly beneficial.

The combination of Wild or Bear's Garlic (Allium ursinum) with ordinary cultivated garlic (Allium sativum) exerts a remarkable synergism in effect upon blood lipids. As can be seen in one of the Examples, the combination of the two species in human patients roughly doubles the expected impact upon total cholesterol levels found in the best results of published records of garlic studies and also much more powerfully regulates triglyceride levels than has been shown with cultivated garlic in the literature. Moreover, this combination proved effective in further reducing these blood lipids even in a patient already being medically treated with a combination of Mevacor® and Lopid®. Similarly powerful effects were found in the results with low-density cholesterol (LDL) and very low density cholesterol (VLDL), benefits being two to times or more that found in the published literature. In other words, there is an obvious and powerful synergism found in the conjoint use of Wild or Bear's Garlic (Allium ursinum) with ordinary cultivated garlic (Allium sativum) and not a mere additive effect.

A further and curious benefit of Wild Garlic when taken in conjunction with high-allicin garlic is that the Wild Garlic reduces the odor-causing potential of the allicin. Wild Garlic, even when consumed in relatively large quantities (it can be eaten as a salad, not merely as a condiment), does not tend to cause breath odor, hence it would be expected that replacing a percentage of cultivated garlic with Wild Garlic would reduce breath odor. Not at all to be expected is that the Wild Garlic itself seems to inhibit the odor-causing potential of allicin. One explanation could be support of liver function with Wild Garlic because of methyl-donating components found in this species, thus improving the liver's ability to detoxify allicin.

Therefore, it has been discovered that the combination of Wild or Bear's Garlic (Allium ursinum) with ordinary cultivated garlic (Allium sativum) and or its allicin/allicin-potential component produces a powerful synergism with regard to reducing elevated blood lipids not previously or elsewhere remarked. Conjoint use of these species delivers, as well, improved blood pressure benefits and improved control of garlic odor. A variety of protective delivery technologies might be expected to further improve the benefits taught here. Known heart protective nutrients and herbs, such as folic acid and red yeast rice, might be combined with the combination of garlics to good effect.

### Methods of Preparing the Combination of Allium ursinum and Allium sativum

Fully realizing the benefits of these two garlic sources and how they might be formulated into a pharmaceutically acceptable formulation has not been previously demonstrated. To avoid the negative effects of allicin, odor, gastrointestinal tract irritation, and so forth as previously noted requires the dosage of an allicin-containing preparation be reduced as much as possible. However, this action risks falling below the therapeutic threshold necessary for reducing dyslipidemia and lowering blood pressure. The actions of garlic that affect lipids, unlike those that influence blood pressure, are best realized via a sustained influence upon the liver. The addition of Allium ursinum to a high allicin garlic Allium sativum preparation will maximize the lipid-lowering effects of allicin, and using polymeric acid retardant pharmaceutical technology will serve to protect allicin from degradation in the stomach. Controlled release technology can be used to support and sustain allicin's lipid-lowering actions in a way not possible with simple enteric coating technology. Special controlled release technology can protect the garlic components from being acted upon in the stomach and yield sustained release in the upper GI-tract, both actions which can promote the realization of the potential of this combination of garlics. Examples are given below.

Prior art in this area not only is quite surprisingly weak, but sometimes egregiously counterproductive. U.S. Pat. No. 6,270,803 (Blatt et al., 2001, “Controlled release garlic formulations”) is an example of a counterproductive approach to controlled delivery. Here is claim 2:
An orally-administrable formulation for the controlled release of granulated garlic according to claim 1, comprising microencapsulated granulated garlic particles which have been microencapsulated by direct coating with an enteric coating and at least one pharmaceutically acceptable diluent, adjuvant or excipient therefor, characterized in that the total in vitro dissolution time of said formulation required for release of 75% of the Allicin available from said formulated based upon the total amount of alliin initially present in said formulation is between about 4 and about 18 hours, as determined by the U.S.P. XXIII paddle method at a paddle speed of 150 rpm, using simulated intestinal fluid without the digestive enzymes normally found in intestinal fluid, containing 0.1% w/w sodium dodecyl sulfate (SDS), at pH 6.8, and a temperature of 37.degree. C.

The problem with this art taught by Blatt et al. is obvious to anyone skilled in gastrointestinal physiology. Fifty percent emptying of the small intestine takes place in 2.5 to 3 hours. (Camilleri M, Colemont L J , Phillips S F, et al. Am J Physiol Gastrointest Liver Physiol. 1989;257:G284-G290.) This assumes a solid meal of typical size. Smaller meals, more liquid meals, meals with high-salt loads, etc., will empty faster. Meals given to diabetics and individuals with other conditions, depending upon the condition and even the stage of the condition, may empty either more quickly or more slowly.

Drug absorption in the gastrointestinal tract is limited by GI transit time and the high rate of mucus turnover. Blatt et al. teach art in which only 75 percent of the payload of the garlic formulation has been released, as best, at 4 hours, which is to say that the preponderance of the active constituents from the garlic is not being released into the portion of the alimentary canal from which it can be absorbed. This is especially true of the water-soluble components of garlic, including the fractions responsible for lowering blood pressure and the adenosine (of Allium ursinum). Nutrients known to be reliably absorbed below the ileum are potassium, sodium chloride, water, a small amount of vitamin K from colonic bacteria, and short chain fatty acids and volatile fatty acids produced by fiber fermentation. Unless specifically demonstrated otherwise, most other nutrients are absorbed above the large intestine. It should be noted, as well, that Blatt et al., moreover, are unaware of the concept of mucoadhesives. As a method for preserving or improving uptake of the actives from garlic, the method taught by Blatt et al. is an obvious failure. In point of fact, mucosal adhesives arguably should be used with all controlled, delayed or sustained-release formulations to improve the predictability of results.

Biodegradable Polymers

A large variety of compounds can be used to create acceptable controlled delivery formulations suitable for use with Allium sativum and Allium ursinum. Hydrophobic components should prevent release in the stomach. However, the target for complete disintegration within the small intestine should be approximately 3 hours. This avoids the initial dumping of components while at the same time allowing for more or less total uptake of the ingredients before intestinal emptying into the colon.

Those polymers which can be used for entrapment of medicinal agents, cover a broad scope of polymeric materials. The most common polymers have been co-glycolides of lactic and glycolic acid. These polymers are usually used as admixed in specific ratios. The following are biodegradable polymers that can be used to entrap medicinal agents that can be administered eitherorally or parenterally: Poly (caprolactone) PCL, Poly (glycolic acid) PGL, Poly (DL-Lactic acid) PDLA, Poly (Lactide-co-glycolide) PLG, (−) Poly (3-hydroxybutyric acid) PHB, (−) Poly (3-hydroxybutyric acid 3-hydroxyvaleric acid) PHBV, chitosan, pullulan, zein, alginic acid and alginate salts. In addition to these biodegradable polymers, other polymers of methacrylate can be used in both organic and water phase methods. These include material made from polymerization and commercialized by Rhöm Pharma of Germany and include, but are not limited to, the following items: acrylic acid and methacrylic acid copolymers, methyl methyolacylate copolymers, ethoxyethyl methacrylate, methyl methacrylate, methyl methoxyacrylate copolymer, aminoalkyl methacrylate copolymers, methacrylate acid copolymer, polyacrylic acid/polymethacrylic acid, methacrylic acid alkylkamide copolymer, polymethacrylic acid (anhydride), polymethyl methacrylate. The methacrylates can be used in the organic coacervation heat derived method or can be used in the water phase pressure evaporation process with heat.

The formulations of this invention retards the release of the active drug or drugs in the gastric juice via the acid retardant polymer coating. These polymers slowly dissolve in the alkaline media of the small intestine. At this time the hydrogel matrix becomes hydrated and swells producing a diffusion membrane which further slows the solvation rate of the active drugs and their subsequent absorption. This hydrogel matrix serves as a secondary membrane to control the diffusion of the active agent from the tablets.

Additionally, the active agents, their pharmaceutical salts or different forms can be granulated into two or more fractions. The first fraction would contain active material coated with a polymer which would release more quickly than the second or third coat. Such a polymer could be polyvinyl acetate phthalate, hydroxypropylmethyl cellulose phthalate, cellulose trimellitate acetate, or methacrylate copolymers. The first fraction will contain plasticizer appropriate for the polymer being used and the polymer strength will be 1-15 weight percent and more preferably 1-13 weight percent. The second fraction would contain a polymer that would release in a more alkaline media. These polymers would be cellulose acetate phthalate, low permeability methacrylate copolymers, ethylcellulose or zein. These polymer coats would range from 1-10 weight percent and preferably be between 3-8 weight percent. The fractions coated may be equal in amount or the ratio may be varied depending upon the delivery pattern to be achieved.

Turning to the preferred process of the present invention, all materials are weighed and screened through a number #93 mesh sieve to insure uniformity of particle size. The preferred method of granulation is with a fluidized bed dryer. The active agents and excipients are blended in the fluid bed dryer for fifteen minutes at a temperature of 40° C. The polymer coating solution is then sprayed into the fluid bed with continuous fluidization at 40° C. and at an atomizing pressure of 0.5-1 bars and a spray rate of 2 grams per minute. The spraying rate may be changed depending upon the degree of hydration observed in the material being granulated. In most instances, aqueous solvent systems are preferable. When an organic solvent is used, the polymer volatilization rate may be faster and subsequently the infusion rate of the polymer may be increased. Plasticizer used for the polymers may be any accepted agents such as glycerin, triacetin, diethyl phthalate, dimethyl phthalate, polyethylene glycol, etc. The amount of plasticizer per weight of polymer may range from 0.25-5 weight percent but is more preferably 0.5-2 weight percent.

Mucoadhesives (bioadhesives) prolong the residence time at the site of absorption thus increasing the period over which the drug can be absorbed and increase the uptake of a number of high molecular weight compounds. Bioadhesives can include any number of substances. Certain carbohydrates, plant lectins, bacterial adhesins, and antibodies can also be used for site-specific mucoadhesion. Some carbohydrate containing copolymers show association with intestinal tissue. Galactose-containing copolymers show a high affinity towards the duodenum and the first part of the jejunum, whereas fucose-containing copolymers have a higher affinity for the last part of the jejunum. Fucosylamine containing copolymers, such as HPMA, are selective to the colon and are used in the delivery of 5-ASA in the case of inflammatory bowel disease. Cationic derivatives bind strongly to all parts. In general use, mucoadhesives are preferably Carbomer® in the range of 1-25% and Methocel K4M® in the range of 1-25%, but others may be used alone or in combination, such as pectins, alginates, and other known and described mucoadhesives, biologically safe agents commonly used in the art.

### SUMMARY OF THE INVENTION

The inventor has discovered that Wild Garlic (Bear's or Forest Garlic, Allium ursinum) can be combined with cultivated high-allicin garlic (Allium sativum) and extracts of the same to take advantage of the synergism of preparations made from these two garlic species. There is a substantial cost savings over the use of pure, Wild Garlic and an elimination of the side effects found with the chronic ingestion of high levels of cultivated allicin-rich garlic (Allium sativum). The resulting combination has demonstrated benefits with regard to blood lipids regulation in individuals in need thereof that are two or more times that expected from the use of either garlic species taken singularly. Blood pressure regulating benefits are maintained or improved, the odor-causing potential of high-allicin garlic is reduced, and significant improvements are found in. the areas of total, LDL, and VLDL cholesterol and triglycerides, benefits far beyond the merely additive. HDL cholesterol levels also are improved. It is to be expected that these benefits, in addition, can be extended through the concurrent intake of folic acid, red yeast rice extracts and other compounds proposed for improving cardiovascular health. Still further, a variety of defined delivery approaches, including, but not limited to controlled releases, especially those including mucoadhesive, can be utilized to improve the effects taught herein.

### DESCRIPTION OF THE PREFERRED EMBODIMENTS

Both Wild Garlic (also known as Bear's or Forest Garlic, Allium ursinum) and cultivated high-allicin garlic (Allium sativum), as well as extracts of the same, have been shown to be active when consumed orally in clinical and animal studies. Therefore, the invention taught herein can be realized without resort to special delivery systems, albeit the garlic combination will benefit markedly from such preparations, especially those including mucoadhesive. As far as the ratios of the garlics is concerned, although Wild Garlic need not be the preponderant ingredient, neither can it reasonably be a trivial component. The exact ratio required to take advantage of the adenosine content of the Wild Garlic, however, will depend upon the delivery used and, perhaps, other ingredients included in the formula. In practice, an effective range of intake of Wild Garlic runs from 100 mg to 1,500 mg per day, keeping in mind that Wild Garlic, unlike cultivated garlic, can be consumed even as a salad vegetable. The amount of allicin-rich garlic or garlic extract or its equivalent in terms of allicin potential ranges from 100 mg to 1500 mg per day.

Higher intakes of the latter are not recommended due to the odor and mild toxicity found with excessive intakes of allicin or its equivalent. Controlled delivery allows once daily dosage.

In controlled delivery formulation for the product, a disintegration time of 3 hours is considered to be optimal as tested using a pH 6.8 monophosphate buffer at 0.05 M concentration under simulated intestinal conditions. The acceptable range for complete disintegration is 45 min to 3 hours ±30 minutes; the presence of a mucoadhesive means that the full payload of the granulate can reliably be delivered even with unusually rapid motility or poor uptake from the small intestine. Formulations undergoing disintegrating at 4 hours or longer offer extremely poor uptake without inclusion of one or more mucoadhesives under supervision of one highly skilled in the art. It should be noted that β-Hydroxypropylcyclodextrin and cyclodextrins in general, given in some examples below, although helpful for improving the uptake of many drugs, are not by themselves adequate controlled release components, nor are they made so by the addition of normal coating materials, nor do they act as mucoadhesives.

### EXAMPLE 1

| Ingredients | Percent | Mg Per Capsule |
|---|---|---|
| Wild Garlic, Allium ursinum | 48.78% | 200.00 |
| Cultivated Garlic, Allium sativum | 48.78% | 200.00 |
| Cellulose | 1.21% | 5.00 |
| Magnesium Stearate | 0.96% | 4.00 |
| Silica | 0.24% | 1.00 |
| Total | 99.97% | 410.00 |

In this simple formula, the first two ingredients, the two garlics, are mixed in standard blending equipment until evenly blended. The last three ingredients are pre-blended and then added to the larger mixture of the garlics and again blended until uniformly dispersed. The resulting powder is encapsulated at the rate of 410 mg per capsule. Daily intake would be two to four capsules, meaning 800 mg to 1,600 mg of the garlic combination per day, with the product being taken in at least two daily dosages. With the addition of suitable binding agents, such as dicalcium phosphate, the blend of this example can easily be used in tablet manufacture. Red yeast rice extract or other similar actives can be added with benefit.

### EXAMPLE 2

| | | | |
|---|---|---|---|
| Ingredient | mg/tablet | Percentage | Amount/100 kg |

| | | | | | | |
|---|---|---|---|---|---|---|
| 1. | Allium sativum | 400.00 | mg | 40.0% | 40.00 | Kg |
| 2. | Allium ursinum | 400.00 | mg | 40.0% | 40.00 | Kg |
| 3. | Avicel ® PH 101 | 105.00 | mg | 10.0% | 10.50 | Kg |
| 4. | Carbopol ® 974-P | 20.00 | mg | 20.0% | 2.00 | Kg |
| 5. | Carbowax ® 3335 | 45.00 | mg | 45.0% | 4.50 | Kg |
| 6. | Starch 1500 | 10.00 | mg | 10.0% | 1.00 | Kg |
| 7. | Cellulose Acetate | 5.00 | mg | 0.5% | 0.50 | Kg |
| | Phthalate | | | | | |
| 8. | Cellulose Acetate | 5.00 | mg | 0.5% | 0.50 | Kg |
| | Trimalleate | | | | | |
| 9. | Magnesium Stearate | 10.00 | mg | 1.0% | 1.00 | Kg |

| | | | | | |
|---|---|---|---|---|---|
| TOTAL | 1,000.00 | mg | 100.00% | 100 | Kg |

Directions:
1. Screen and mix the garlic's with Avicel®, Carbopol® and Carbowax® in a Hobart blender and thoroughly for 5 minutes at sped 1.
2. Dissolve CAP and CAT in 1000 mL of deionized water to which 29% ammonia hydroxide has been added. Blend in a high shear mixer until the solution is clear.
3. Pour the CAP and CAT mixture into the blending powders slowly until an overwet granulation forms, then blend for 3-4 minutes.
4. After the material is thoroughly mixed and blended turn out on paper lined trays and allow evaporation of the solution to occur over the next 24 hours. When a hard granulate has formed, remove from the trays and pass through a Fitzmill comunitor with knives forward at medium speed and use an 093 screen.
5. Place the granulate into the Hobart and bend with magnesium sterate for 1-2 minutes. Remove the granulate and compress into oblong tablets with a weight of 1,000 mg and a hardness of 15-20 kgs facture force strength.

6. In a water bath of pH 6.8 and 0.05M KH2PO4, they should have a disintegration time of 3 hours ±30 minutes.

### EXAMPLE 3

| Ingredient | mg/tablet | Percentage | Amount/100 Kg |
|---|---|---|---|
| 1. Allium sativum | 400 | 40.0000 | 40.0000 |
| 2. Allium ursinum | 400 | 40.0000 | 40.0000 |
| 3. Dibasic Calcium Phosphate | 70 | 7.0000 | 7.0000 |
| 4. Carbopol ® 974-P | 19.1 | 1.9100 | 1.9100 |
| 5. Carbowax ® 3335 | 30.0 | 3.0000 | 3.0000 |
| 6. Folic Acid | 0.40 | 0.0400 | 0.0400 |
| 7. Pyridoxine | 50.0 | 5.0000 | 5.0000 |
| 8. Vitamin B12 | 0.50 | 0.0100 | 0.0500 |
| 7. Starch 1500 | 10.0 | 1.0000 | 1.0000 |
| 8. Cellulose Acetate Phthalate | 10.0 | 1.0000 | 1.0000 |
| 9. Magnesium Stearate | 10.0 | 1.0000 | 1.0000 |
| TOTAL | 1,000 | 100.00 | 100.0 |

Directions:
1. Mix in large Hobart or Bohle granulator items 1-8 and blend for 5 minutes.
2. Dissolve the cellulose acetate phthalate in deionized ammoniated water using a high shear blender.
3. Granulate the blended items with the ammoniated CAP 1,000 mL so as to form an overwet granulation.
4. Remove the granulate from the machine and place it into a fluid bed dryer where it should be dried at 40° C. When the granulate is dry as determined by a loss on drying analysis (LOD) reduce the granulate in size by running it through a D3 Fitzmill comunitor using a #93 screen with knives forward at moderate speed.
5. When the granulate is free flowing and dry blend with magnesium sterate for 5 minutes. Then transfer the granulate to a tableting machine and compress the material into capsule shaped punches to a weight of 1,000 mg and a fracture force strength of 15-20 kg. When finished with the tableting, place the tablets into a pan coater and coat the individual tablets with a 3% solution of opadry film coating solution.
6. The finished tablets can then be packaged in HDPE light opaque containers and labeled.
7. The finished tablets should have a disintegration rate of about 3 hours using pH 6.8 monophosphate buffer at 0.05 M concentration.

In this mixture the concentration of Allium sativum and Allium ursinum may range from 50mg-1,000 mg as individual items. The formulation provides a controlled release of the active compounds or any pharmaceutically acceptable extract thereof. The drug particles are coated along with excipients with 0.5-20 weight percent of an acid resistant polymer, or hydrophobic polymer preferably from 1-20 weight percent. The polymer coated granules are incorporated into a hydrogel matrix. The polymer gel comprises 1-25 weight percent of the formula and preferably 0.5-20 weight percent. The amount of vitamins added are within the safe and daily accepted range for use by an adult.

Variations of this basic formula can be created by one skilled in the art. The preferred hydrogel polymers are carboxypolymethylene an acrylic acid polymer (carbomer) sold under the mark Carbopol-974-P® by Noven. These polymers are present in amounts of 0.5-20 weight percent and preferably from 5-20 weight percent. Additionally the second polymer used is a high viscosity of hydroxypropylmethyl cellulose derivative sold as Methocel 4KM-P® by Dow Chemical Corporation. This polymer is present in amounts of 1-20 weight percent and preferably from 3-15 weight percent. The formulation additionally is composed of bulking agents, disintegrants and lubricants.

The preferred acid resistant polymers used for coating the drug granules include, but are not limited to cellulose acetate phthalate, hydroxypropylmethyl cellulose phthalate, polyvinyl acetate phthalate, cellulose acetate trimellitate, methacrylate copolymers in various forms sold as Eudragit®. Additionally other agents may be used which are hydrophobic and not acid resistant such as ethylcellulose, zein, and certain methacrylate copolymers. The compressed tablet may be film coated with acid resistant or hydrophobic film coating agents. Such polymers may include coloring pigments as well as plasticizer.

### EXAMPLE 4

| Ingredient | | mg/tablet | Percentage | Amount/kg |
|---|---|---|---|---|
| 1. | Allium sativum | 400 | 40.0% | 40.0000 |
| 2. | Allium ursinum | 400 | 40.0% | 40.0000 |
| 3. | Dibasic Calcium | 60 | 6.0% | 6.0000 |
| | Phosphate | | | |
| 4. | Carbopol ® 974-P | 19.1 | 1.9% | 1.9000 |
| 5. | Carbowax ® 3335 | 20.0 | 2.0% | 2.0000 |
| 6. | Folic Acid | 0.40 | 0.04% | 0.0400 |
| 7. | Pyridoxine | 50.0 | 5.0% | 5.0000 |
| 8. | Vitamin B12 | 0.50 | 0.10% | 0.0500 |
| 9. | Starch 1500 | 10.0 | 1.0% | 1.0000 |
| 10. | Eudragit ® D L 100 | 20.0 | 2.0% | 2.0000 |
| 11. | Eudragit ® S PO | 10.0 | 1.0% | 1.0000 |
| 12. | Magnesium Stearate | 10.0 | 1.0% | 1.0000 |

| | | | | | |
|---|---|---|---|---|---|
| TOTAL | 1,000.0 | mg | 100.00% | 100.0 | Kg |

Directions:
1. In Hobart or Bohle machine place items 1-9 and blend for 3-5 minutes with medium impeller speed.
2. Once the powders are thoroughly mixed slowly spray in the Eudragit® solution which can be a 30% aqueous dispersion or the powders made up in 1,000 mL of deionized water and alkalinized with 4% NaOH.
3. Allow the powders to absorb the polymers until a distinct granulate has formed. At this point remove the granulate from the mixer and place in a fluid bed dryer. Dry at 40° C. until the LOD is less than 2.5%.
4. When the granulate is dry, put the material through a Fitzmill using a #93 screen with knives forward and with moderate speed. Take the granulate and using a capsule shape punch press out tablets with a weight of 1,000 mg and a fracture force of 15-20 kg.
5. Next the tablets should be coated with 3% Opadry® solution in a suitable spray drying pan at 40° C. Other coating agents such as Eudragit® Filmtab® or similar agents known in the art may be used. This film coating is to cover the tablets odor and appearance.
6. After film coating the tablets they should be tested for disintegration in pH 6.8 monophophate buffer. The disintegration time should be 3 hours ±30 minutes.

### EXAMPLE 5

| Ingredients | mg/tablet | Percentage | Amount/5 Kg | |
|---|---|---|---|---|
| 1. Allium sativum | 400 | 40.0% | 2.0000 | |
| 2. Allium ursinum | 400 | 40.0% | 2.0000 | |
| 3. Dibasic Calcium Phosphate | 100 | 10.0% | 0.5000 | |
| 4. Carbopol ® 974-P | 35.0 | 3.5% | 0.1800 | |
| 5. Methocel ® K4M | 20.0 | 2.0% | 0.1000 | |
| 6. Hydroxypropylcyclodextrin | 5.0 | 0.5% | 0.0200 | |
| 7. Strach 1500 | 10.0 | 1.0% | 0.0500 | |
| 8. Cellulose Acetate Phthalate | 20.0 | 2.0% | 0.1000 | |
| 9. Magnesium Stearate | 10.0 | 1.0% | 0.0500 | |
| TOTAL | 1,000 | 100% | 5.000 | kg |

Directions:
1. Into Hobart or Bohle granulator place items 1-5+7 and blend for 2-3 minutes.
2. After blending is complete spray in item 6 which has been dispersed into purified water, 200 mL. Continue blending until powders are dry. Then disperse CAP, item 8 into 500 mL of ammoniated water with a high shear mixer. Spray the CAP into the wetted mass until there is a distinct granulate that forms. If necessary use choppers to further disperse the granulate. When the granulate has an LOD of <2.5% remove from the granulator and continue drying in fluid bed dryer at 40° C. until the granulate is dried to <1% LOD.
3. Remove the granulate and reduce in size by passing through a #93 screen on a Fitzmill cominutor. Take this granulate and blend in a suitable device with magnesium sterate added, for 1-2 minutes. 4. After the granulate has been blended with the magnesium stearate, place into a rotary press with capsule shaped punches and compress tablets into tablets with a weight of 1,000 mg and a fracture force of 15-20 kg. Following the tableting, take the tablets to the film coating area and spray coat the tablets with an opaque water soluble film of 3%. After the tablets have been coated and polished they are ready for packaging. Disintegration will be in the range of 3 hours.

### EXAMPLE 6

| Ingredient | | mg/tablet | Percentage | Amount/100 Kg |
|---|---|---|---|---|
| 1. | Allium sativum | 400 | 40.0000 | 40.0000 |
| 2. | Allium ursinum | 400 | 40.0000 | 40.0000 |
| 3. | Dibasic Calcium | 80 | 8.0000 | 8.0000 |
| | Phosphate | | | |
| 4. | Carbopol ® 974-P | 19.1 | 1.9100 | 1.9100 |
| 5. | Methocel ® K4M | 10.0 | 1.0000 | 1.0000 |
| 6. | Folic Acid | 0.40 | 0.0400 | 0.0400 |
| 7. | Pyridoxine | 50.0 | 5.0000 | 5.0000 |
| 8. | Vitamin B12 | 0.50 | 0.0500 | 0.0500 |
| 7. | Starch 1500 | 10.0 | 1.0000 | 1.0000 |
| 8. | β-Hydroxypropyl- | 10.0 | 1.0000 | 1.0000 |
| | cyclodextrin | | | |
| 8. | Cellulose Acetate | 10.0 | 1.0000 | 1.0000 |
| | Phthalate | | | |
| 9. | Magnesium Stearate | 10.0 | 1.0000 | 1.0000 |

| | | | | | |
|---|---|---|---|---|---|
| TOTAL | 1,000 | mg | 100.00% | 100.00 | Kg |

Directions:
1. Into Hobart or Bohle granulator add items 1-7 and blend with slow steady speed for 5 minutes.
2. Mix beta-hydroxypropylcyclodextrin into 100 mL purified water with low heat and constant stirring. When finished spray the powders with the solution and continue blending until little moisture is present.
3. Into 1,000 mL of ammoniated water add cellulose acetate phthalate with vigorous stirring. Blend until clear, then spray into powders that have been coated with cyclodextrin.
4. Remove the granulate and place into fluid bed dryer with the inlet temperature set at 40° C. and continue blending until the LOD is <2.0%. At that point remove the granulate and reduce in size through a communitor using a #93 screen. After size reduction add magnesium stearate and blend for about 5-7 minutes.
5. After the granulate has been blended with the magnesium stearate, place into a rotary press with capsule shaped punches and compress tablets into tablets with a weight of 1,000 mg and a fracture force of 15-20 kg.
6. Following the tableting take the tablets to the film coating area and spray coat the tablets with an opaque water-soluble film of 3%. After the tablets have been coated and polished they are ready for packaging.
7. Check the tablets for disintegration they should be at pH 6.8 in monophophate buffer at 0.05 M and complete disintegration should occur within 3 hours ±30 minutes.

### EXAMPLE 7

Examples 2-6 provide controlled delivery formulations designed to protect the garlic combination from exposure to gastric fluids while allowing release into the small intestine. These basic formulations and variations thereof can be used to create pre-mixes to which other ingredients not requiring controlled delivery can be added and the whole with appropriate excepients, pressed into tablets. In such cases, the tablet is a combination of immediate and controlled release components. Typical additional ingredients benefiting cardiovascular health and not requiring controlled delivery would include trivalent chromium compounds, Coleus forskohlii extracts, grape seed and skin extracts, guggulsterones, hawthorne berry and flower extracts, pomegranate extract, red yeast rice extract, ubiquinone and zinc. High-dose plant sterols used to interfere with the absorption of dietary cholesterol should not be delivered in conjunction with the art taught herein due to likely interference with the uptake of the active ingredients of the garlic components.

### EXAMPLE 8

| Ingredient | Wt (Kg) | Percentage |
|---|---|---|
| 1. Allium sativum | 1.000 | 45.45 |
| 2. Allium ursinum | 1.000 | 45.45 |
| 3. Magnesium Stearate | 0.200 | 9.09 |
| TOTAL | 2.20 | 99.99% |

Directions:
1. Using a Bohle VMA-20 granulator, add items 1-3 and blend for 5 minutes.
2. The material in step 1 next is heated to approximately 35° C. while bending is continued. This step is continued long enough to melt the magnesium stearate and to coat the mixture evenly.
3. Contents are discharged to separate bowl for cooling.
4. The resulting granulate is screened through a 093 D3 Fitzmill screen to control the size of the particles.
5. One or more mucoadhesives can be dry mixed with the granulate and under compression will become part of the matrix.
   Magnesium stearate is insoluble in water and only slowly decomposed by dilute acids. Accordingly, this simple melt operation produces a garlic granulate that is resistant to stomach fluids, but releases quickly when exposed to bile and other fluids of the small intestine. Through the use of di-calcium phosphate and other known and inexpensive tableting agents, mucoadhesive(s), etc., it is convenient for one skilled in the art to create tablets that will disintegrate completely within 45-180 minutes of entering the small intestine.

### EXAMPLE 9

| Ingredient | Wt (Kg) | Percentage |
|---|---|---|
| 1. Allium sativum | 2.000 | 55.55 |
| 2. Allium ursinum | 1.000 | 27.78 |
| 3. Lubritab ® (hydrogentated vegetable oil) | 0.600 | 16.67 |
| TOTAL | 3.600 | 100.0% |

Directions:
1. Placed the two garlics and the Lubritab® in Bohle VMA-20 bowl. (Lubritab® tends to cake and requires prescreening.)
2. Set temperature to 69° C.; impeller speed is set to 50 RPM.
3. Contents are mixed under vacuum until product temperature reached 57-58° C. Total blending time is approximately 20 minutes.
4. Contents are discharged to separate bowl for cooling.
5. The resulting granulate is screened through a 093 D3 Fitzmill screen to control the size of the particles.
6. One or more mucoadhesives can be dry mixed with the granulate and under compression will become part of the matrix.
   This operation may substitute room temperature solid fractions derived from coconut, palm, sesame, soy and other oils for Lubritab®. Lubritab® is often used in direct compression wax matrix operations to create controlled delivery tablets, but the amounts required are 20-40 percent of the total tablet weight and the protection is less good than that afforded at 10-15 percent via a melt operation. Oils/waxes with lower melting points are preferred as long as they remain solid at room temperature, sesame fractions being particularly desirable. Through the use of di-calcium phosphate and other known and inexpensive tableting agents, mucoadhesive(s), etc., it is convenient f one skilled in the art to create tablets that will disintegrate completely within 45-180 minutes of entering the small intestine. The addition of non-coated active components to tablets, such as red yeast rice extract, can be accomplished without loss of benefit.

### EXAMPLE 10

| Ingredient | Wt (Kg) | Percentage |
|---|---|---|
| 1. Allium sativum | 2.000 | 55.55 |
| 2. Allium ursinum | 1.000 | 27.78 |
| 3. TPGS | 0.600 | 16.67 |
| 3. Isopropyl Alcohol (99%) | 0.600 | |
| TOTAL (solids) | 3.600 | 100.00% |

Directions:
1. Weigh d-alpha-tocopheryl polyethylene glycol succinate (TPGS) and place in beaker.
2. Heat on hot plate at approximately 40° C. until melted.
3. Add alcohol slowly to TPGS with stirring until fully mixed to yield a clear yellow liquid.
4. Place garlics in Bohle VMA-20 bowl with heat setting of 40° C.
5. Set Impeller rate to 50 RPM and warm product to approximately 37° C.
6. Vacuum TPGS solution into VMA-20 bowl via spray nozzle (approximately 5 minutes).
7. Set vacuum to 850 mbar and mix product with vacuum drawing solution until all is used.
8. Transfer the resulting mixture to a fluid bed dryer.
9. Dry at 29.7° C. until material is visibly dry as well as loose to the touch.
   Through the use of di-calcium phosphate and other known and inexpensive tableting agents, mucoadhesive(s), etc., it is convenient for one skilled in the art to create tablets that will disintegrate completely within 45-180 minutes of entering the small intestine. The addition of non-coated active components to tablets, such as red yeast rice extract or folic acid, can be accomplished without loss of benefit.

### EXAMPLE 11

Baseline

| | | | | | | | Triglyc- | Other |
|---|---|---|---|---|---|---|---|---|
| Pt. | Age | Sex | TC | LDL | VLDL | HDL | erides | Meds |
| JD | 65 | M | 235 | 100 | 120 | 27 | 225 | Mevacor ® |
| | | | | | | | | Lopid ® |
| LB | 35 | M | 254 | 120 | 145 | 35 | 185 | None |
| BT | 58 | F | 275 | 110 | 135 | 42 | 167 | None |
| LV | 35 | M | 256 | 125 | 140 | 44 | 180 | None |
| BH | 66 | M | 275 | 115 | 155 | 35 | 190 | None |

After 6 Weeks Garlic Therapy

| | | | | | | | Triglyc- | Other |
|---|---|---|---|---|---|---|---|---|
| Pt. | Age | Sex | TC | LDL | VLDL | HDL | erides | Meds |
| JD | 65 | M | 122 | 44 | 75 | 35 | 130 | Mevacor ® |
| | | | | | | | | Lopid ® |
| LB | 35 | M | 153 | 45 | 60 | 47 | 140 | None |
| BT | 58 | F | 222 | 65 | 75 | 40 | 175 | None |
| LV | 35 | M | 146 | 55 | 80 | 48 | 130 | None |
| BH | 66 | M | 157 | 60 | 83 | 43 | 125 | None |

Percentage Change In Lipids

| | | | | | | | | Other |
|---|---|---|---|---|---|---|---|---|
| Age | Pt. | Sex | TC | LDL | VLDL | HDL | Triglycerides | Meds |
| 65 | JD | M | −48.1% | −66% | −37.5 | +22.9% | −42.2% | Mevacor ® |
| | | | | | | | | Lopid ® |
| 35 | LB | M | −39.2% | −45.0% | −58.6% | +34.3% | −24.3% | None |
| 58 | BT | F | −19.3% | −37.2% | −44.4% | +4.8% | −4.3% | None |
| 35 | LV | M | −43.0% | −56.0% | −42.9% | +8.3% | −27.8% | None |
| 66 | BH | M | −43.0% | −43.5% | −46.5% | +18.6 | −36.8% | None |

| | | | | | |
|---|---|---|---|---|---|
| Mean Change | −38.5% | −49.5% | −45.4% | +17.8% | −27.2% |

Tablets produced according to the method of Example 2 were tested with five subjects as indicated in the chart above, one tablet per day with a meal, by James M. Dunn, MD. The Wild Garlic, 400 mg, supplied not less than 2 mg/gram adenosine. The high-allicin garlic, 400 mg, supplied 11,000 mcg alliin, the equivalent of 5,000 mcg allicin activity. The results seen here are far above the best that have been reported in clinical trials with comparable initial readings, e.g., the 25 percent reduction in total cholesterol mentioned by Sendl, et al. In clinical trials, results with garlic and garlic extracts generally have been disappointing. Most clinical work has shown reductions of total cholesterol of less than 16 percent even after 12 to 24 weeks of treatment. Stevenson C, Pittler M H , Ernst E. Garlic for treating hypercholesterolemia. A meta-analysis of randomized clinical trials. Ann Intern Med. 2000 Sep. 19;133(6):420-9.) Moreover, enteric coating high-allicin regular garlic has not been shown to dramatically improve results. One recent published trial found only a 4.2% reduction in total cholesterol and a 6.6% reduction in LDL cholesterol, whereas HDL cholesterol actually declined 0.9% versus a 9.1% increase found with placebo. (Kannar D, Wattanapenpaiboon N, Savige G S, Wahlqvist M L. Hypocholesterolemic effect of an enteric-coated garlic supplement. J Am Coll Nutr 2001;20(3):225-31.) Therefore, the results at 6 weeks found with the combination of garlics formulated as in Example 2 constitute a compelling argument that this combination is synergistic.

Authorities consistently have maintained that these two garlic species can be substituted for one another, but not that they would have even an additive effect, let alone a synergistic one. A major difference between the two species is that cultivated garlic is very low in adenosine. High-allicin garlic powder and extracts with high-allicin potential have virtually undetectable levels of adenosine. However, Wild Garlic (Allium ursinum), is quite high in adenosine. Tests on Wild Garlic have indicated an adensosine content ranging between 2500 and 5500 mg/kg. The issue, still unresolved as of this writing, is whether that adenosine is biologically active. Previous pharmacological experience with oral adenosine products would suggest not. A rich source of adenosine that is active when orally ingested is a novel finding in that pure adenosine taken by mouth is poorly absorbed and rapidly metabolized in the red blood cells. Only 5% of orally ingested adenosine typically appears in the blood or tissues. The findings given in this Example indicate that the adenosine supplied by Wild Garlic is, in fact, quite active. This is the first actual, if indirect, proof of this activity via the following line of reasoning: The liver is the organ that primarily controls the levels of fatty acids in the blood. One study has indicated the effects of allicin in lowering cholesterol synthesis are highly dependent upon the availability of adenosine in the system. ([Gebhardt R. Nutrition 1997;13:379-381.) Our finding is that a relatively small amount of Wild Garlic, which is expensive and in short supply because not subject to cultivation, can be combined with an allicin-rich extract of the abundant and inexpensive cultivated garlic to significantly improve the lipid-lowering effect of the allicin. This finding suggests that the adenosine found in Allium ursinum is absorbed and biologically active.

I claim:

1. A composition for facilitating a reduction in blood lipids in an individual in need of such an effect wherein said composition comprises:
(i) a first component consisting of Allium sativum (cultivated or kitchen garlic) in a pharmaceutically acceptable form, and
(ii) a second component consisting of Allium ursinum (also known as Wild, Forest or Bear's Garlic) in a pharmaceutically acceptable form,
wherein said composition is comprised of said first component and said second component in a weight ratio of from 15:1 to 1:15.

2. The garlic-containing compound of claim 1 wherein the composition is formulated as tablets or granulate wherein the amount of Allium ursinum or an extract of the same in a daily dose ranges from 100 mg to 1500 mg and the amount of Allium sativum or an extract of the same ranges from 100 mg to 1500 mg.

3. The garlic-containing compound of claim 1 wherein the composition is formulated with one or more absorption-enhancing/controlled release agents.

4. The garlic-containing compound of claim 1 wherein the composition is formulated with one or more rate-controlling excipients.

5. The garlic-containing compound of claim 1 wherein the composition is formulated with one or more mucoadhesives (bioadhesives) to allow adherence of the active principles to the gastrointestinal wall.

6. The garlic-containing compound of claim 1 wherein the composition is formulated with one or more lubricants.

7. The garlic-containing compound of claim 3 wherein the one or more absorption-enhancing/controlled-release agents are selected from the group consisting of: d-alpha-tocopheryl polyethylene glycol succinate (TPGS); Lubritab®; volcanic oils suitable for melt operations; high viscosity grades of conjugated polyethylene glycol; ethylcellulose, carboxymethylcellulose, cellulose propionate; cellulose acetate propionate; cellulose acetate butyrate; cellulose acetate phthalate (CAP); cellulose triacetate; hydroxypropyl-methylcellulose phthalate; polymethyl methacrylate; polyethyl methacrylate; polybutyl methacrylate; polyisobutyl methacrylate; polyhexyl methacrylate; polyisodecyl methacrylate; polylauryl methacrylate; polyphenyl methacrylate; polymethyl acrylate; polyisopropyl acrylate; polyisobutyl acrylate; polyoctadecyl acrylate; polyethylene; polyethylene low density; polyethylene high density; polypropylene; polyethylene oxide; polyethylene terephthalate; polyvinyl isobutyl ether; polyvinyl acetate; polyvinyl acetate phthalate; polyvinyl chloride; polyurethane; other copolymers of acrylic and methacrylic and esters; waxes; shellac; zein; hydrogenated vegetable oils; non-hydrogenated vegetable oil fractions suitable for melt operations and compressions; magnesium stearate; polyvinyl alcohol; polyvinylpyrrolidone; methyl cellulose; hydroxypropyl cellulose; hydroxpropylmethyl cellulose or polyethylene glycol; or a mixture thereof.

8. The garlic-containing compound of claim 3 wherein the one or more absorption-enhancer/controlled-release agents are present from about 0.5% to about 60% of the total weight of the garlic composition of claim 1.

9. The garlic-containing compound of claim 3 wherein the one or more absorption-enhancer/controlled-release agents are present from about 1.0% to about 30% of the total weight of the garlic composition of claim 1.

10. The garlic-containing compound of claim 4 wherein the one or more rate-controlling excipients are selected from the group consisting of: Eastacryl; Kollicoat® (polyvinylalcohol-polyethyleneglycol graft-copolymer); cellulose acetate phthalate; Kollicoat® SR; ethyl cellulose; Eudragit® (family of acrylate and methacrylate-based coatings); zein (vegetable protein); acrylic polymers; polyvinyl acetate phthalate; hydroxymethylpropylmethyl cellulose phthalate; cellulose acetate trimalleate; acrylic polymer plasticizers; polymers of polylactic acid; polymers of glycolic acid, and mixtures thereof; Primogel; Pruv™ (stearyl fumarate sodium); magnesium stearate; citrate esters; triethyl citrate; propylene glycol; and dibutyl sebacate.

11. The garlic-containing compound of claim 4 wherein the wherein the one or more rate-controlling excipients are present from about 0.001% to about 60% of the total weight of the garlic composition of claim 1.

12. The garlic-containing compound of claim 4 wherein the one or more rate-controlling excipients are present from about 0.01% to about 25% of the total weight of the garlic composition of claim 1.

13. The garlic-containing compound of claim 5 wherein the one or more mucoadhesives are selected from the group consisting of: Carbomer®; Methocel K4M®; galactose-containing copolymers; fucosylamine-containing copolymers; pectins; and alginates.

14. The garlic-containing compound of claim 5 wherein the one or more mucoadhesives are present from about 0.01% to about 50% of the total weight of the garlic composition of claim 1.

15. The garlic-containing compound of claim 5 wherein the one or more mucoadhesives are present from about 0.5% to about 25% of the total weight of the garlic composition of claim 1.

16. The garlic-containing compound of claim 6 wherein one or more lubricants are selected from the group consisting of: magnesium stearate, calcium stearate; sodium stearate, glycerol monostearate; stearic acid; Lubritab®; hydrogenated vegetable oils; non-hydogenated vegetable oils and oil fractions; waxes; talc; boric acid; sodium benzoate; sodium acetate; sodium chloride; DL-leucine; sodium oleate; sodium lauryl sulfate; magnesium lauryl sulfate and polyethylene glycols and kaolin.

17. The garlic-containing compound of claim 6 wherein the one or more lubricants are present from about 0.0001% to about 25% of the total weight of the garlic composition of claim 1.

18. The garlic-containing compound of claim 6 wherein the one or more lubricants are present from about 0.01% to about 5% of the total weight of the garlic composition of claim 1.

19. The garlic-containing compound of claim 1 wherein the composition is formulated as tablets or granulate to exhibit a total disintegration time between 45 minutes and 3 hours ±30 minutes as tested using a pH 6.8 monophosphate buffer at 0.05 M concentration under simulated intestinal conditions.

20. The garlic-containing compound of claim 1 wherein the composition is formulated as tablets or granulate and administered to an individual in need of an anti-hypertensive.

21. The garlic-containing compound of claim 1 wherein the composition is formulated as tablets or granulate and co-administered with other serum lipids regulating agents, such as folic acid and red yeast rice extract.
